(19) United States
(12) Patent Application Publication
(10) Pub. No.: US 2005/0189211 A1
(43) Pub. Date: Sep. 1, 2005

(54) Process and apparatus for the conversion of methane gas to higher hydrocarbons

(76) Inventor: Glenn Robert Morton, Spring, TX (US)

Correspondence Address: William B. Miller;Kerr-McGee Corporation, 123 Robert S. Kerr Avenue, Oklahoma City, OK, 73102, US

(21) Appl. No.: 10/786,510

(22) Filed: Feb. 26, 2004

(51) Int. Cl. C07C 1/00

(52) U.S. Cl. 204/157.15

(57) ABSTRACT

A process and apparatus are provided for converting methane to higher hydrocarbons, especially for converting economically stranded natural gas to a transportable liquid hydrocarbon product mainly comprised of C3+ hydrocarbons, comprising applying ultraviolet light to methane (or to the natural gas containing the methane) under conditions effective to cause its photodissociation and polymerization to C2+ and higher hydrocarbons.

### FIELD OF THE INVENTION

The present invention relates to processes for converting methane gas to higher hydrocarbons, and more particularly to processes for converting economically stranded methane gas to higher hydrocarbons which are either liquid at ambient conditions or can be converted to a transportable liquid form with much less effort than implicated with the liquefaction of methane to form liquefied natural gas (or LNG).

### BACKGROUND OF THE INVENTION

In many regions of the world, large natural gas deposits are economically stranded for lacking an economic means for moving the natural gas to market. In this regard, natural gas is predominantly methane, typically being about 90 mol percent or greater methane. In areas where natural gas pipelines are not available to carry the natural gas to market (or where the costs associated with such a pipeline would be prohibitive, as for example in regard to natural gas produced from deepwater offshore wells), the natural gas product must typically be converted to a transportable liquid form to be available for practical use.

Methane, however, is converted to a liquid form only at significant expense. Direct liquefaction of natural gas to form liquefied natural gas (or LNG) requires refrigeration at atmospheric pressure to −260 degrees Fahrenheit (−162 degrees Celsius), and cryogenic and/or high pressure containment systems for maintaining the LNG as a liquid during transport to an offloading facility. Significant capital investments are associated also with the offloading facility for converting the LNG back to gaseous form for further transport in a natural gas pipeline or for use. Further, loading and offloading operations can present a risk of fire and explosion.

A variety of processes have also been proposed for chemically converting the methane in natural gas to higher hydrocarbons which exist as liquids at ambient conditions or which are more readily and economically liquefied. Most of these processes start with the conversion of methane to a synthesis gas comprised of hydrogen and carbon monoxide, followed by the Fischer-Tropsch processing of the syngas to yield transportable higher hydrocarbon liquids or, in some instances, processing of the syngas to yield oxygenates, e.g., methanol. These methods also require significant energy and capital investments, however.

A number of processes have also been proposed which use microwave energy in some fashion to convert methane to higher hydrocarbons. U.S. Pat. No. 5,205,915 to Ravella et al. thus proposes to convert methane to acetylene, ethylene and hydrogen by subjecting the methane to continuous microwave radiation in the presence of at least one plasma initiator capable of initiating an electric discharge in an electromagnetic field, preferably a plurality of metal wire segments. U.S. Pat. No. 5,205,912 to Murphy is essentially comparable, but uses pulsed microwave energy rather than continuous microwave energy. U.S. Pat. No. 5,472,581 to Wan also uses pulsed microwave energy, but in the presence of an activated charcoal catalyst, to convert methane to primarily acetylene. None of these processes have found commercial acceptance, however, and conversions are typically too low to be of interest.

### SUMMARY OF THE INVENTION

The present invention adopts a completely different approach, in providing a process for the conversion of methane gas to higher hydrocarbons which are either liquids at ambient conditions or can be made liquid with conventional refrigeration at atmospheric pressure or not greatly in excess of atmospheric pressure. The process of the present invention more particularly comprises applying ultraviolet light to methane under conditions effective to cause its photodissociation and polymerization to C2+ and higher hydrocarbon products. Per-pass conversion is preferably at least about 45 percent to C2+ and higher hydrocarbons, more preferably is at least about 50 percent and most preferably is at least about 55 percent. In a further step, preferably the products are then cooled to an extent whereby a transportable liquid hydrocarbon product is produced.

The dissociation of methane by ultraviolet light and subsequent hydrocarbon polymerization to higher, C2+hydrocarbons has previously been observed and described in characterizing the atmospheres of Jupiter, Saturn and Neptune, see, e.g, Van Dishoeck and Van Der Hart, “Ab Initio Studies of the Photodissociation Processes in Positive Hydrocarbon Ions. I. The Methane Ion”, Chemical Physics, 50:45-62 (1980); Moses et al., “Photochemistry of Saturn's Atmosphere. I. Hydrocarbon Chemistry and Comparisons with ISO Observations”, Icarus, 143:244-298 (2000); Yelle and Griffith, “Structure of the Jovian Stratosphere at the Galileo Probe Entry Site”, Icarus, 152:331-346 (2001); Moses et al., “Hydrocarbon Nucleation and Aerosol Formation in Neptune's Atmosphere”, Icarus, 99:318-346 (1992). To the inventor's knowledge, however, it has not been suggested that the photochemical phenomena observed in these articles should be applied to economically stranded natural gas produced on this planet.

### DESCRIPTION OF PREFERRED EMBODIMENTS

The process of the present invention preferably involves passing natural gas (optionally being instead natural gas from which non-hydrocarbon impurities such as carbon dioxide and sulfuric acid have been removed or being substantially only methane gas) into a conduit lined with a series of lamps or lasers emitting ultraviolet light characterized by a photon energy of at least about 1.7 electron volts, corresponding generally to photon wavelengths above about 730 nanometers. The UV light is applied under conditions which are effective to cause the photodissociation and polymerization of methane to C2+ and higher hydrocarbon products, preferably with a per-pass conversion of at least about 45 percent of the methane, more preferably at least about 50 percent and most preferably at least about 55 percent. As the cross-section for interaction between the photons and methane molecules is on the order only of 10e-19 square centimeters, preferably the pressure within the conduit will be adjusted to 10 atmospheres gauge or greater. The products are then cooled by conventional refrigeration to yield a transportable liquid hydrocarbon product. Preferably a vapor-liquid separator is used to separate and recycle any unreacted methane from the initial product prior to refrigeration and recovery of the desired transportable liquid hydrocarbon product, which is most preferably comprised of mainly C3+ hydrocarbons.

The UV light may be supplied, for example, by means of a series of barium borate tunable lasers or a set of dielectric discharge excimer lamps. Where desired, various wavelengths of light can be applied through these devices to break single C—H bonds in the methane molecules and mediate subsequent bond breakages, so providing a degree of control over the various C2+ hydrocarbons produced and their relative proportions.

The apparatus employed to carry out the process, including the conduit and associated refrigeration equipment, will preferably be of a size suitable to be installed on an offshore oil and gas production platform or an a floating production storage and offloading (FPSO) vessel, so that the invention is well-suited to application to offshore stranded gas reserves.

An alternate application of the process of the present invention would be for enriching gas entering a conventional natural gas processing plant, to yield greater amounts of the more valuable, chemical feedstock-suitable C2+ hydrocarbons.

1. A process for converting methane to higher hydrocarbons, comprising applying ultraviolet light to methane under conditions effective to cause its photodissociation and polymerization to C2+ and higher hydrocarbons.

2. A process as defined in claim 1, wherein per-pass conversion is at least about 45%.

3. A process as defined in claim 2, wherein per-pass conversion is at least about 50%.

4. A process as defined in claim 3, wherein per-pass conversion is at least about 55%.

5. A process as defined in claim 1, further comprising the step of cooling the C2+ hydrocarbons to provide a transportable liquid hydrocarbon product.

6. A process as defined in claim 5, wherein ultraviolet light is applied to the methane in natural gas.

7. A process as defined in claim 5, wherein ultraviolet light is applied to the methane in natural gas from which non-hydrocarbon impurities have been removed.

8. A process as defined in claim 5, further comprising recovering and recycling unconverted methane prior to cooling the C2+ hydrocarbons to provide the transportable liquid product.

9. A process as defined in claim 1, wherein the ultraviolet light is applied within a conduit lined with a series of devices emitting ultraviolet light having a photon energy of at least about 1.7 electron volts.

10. A process as defined in claim 9, wherein the pressure within the conduit is maintained at 10 atmospheres gauge or greater.

11. A process as defined in claim 1, applied to natural gas produced from an offshore natural gas-producing well.

12. A process as defined in claim 1, applied to natural gas entering a natural gas processing plant.

13. Apparatus for converting natural gas produced from a well site which is too remote to permit economic recovery of the natural gas through an associated pipeline to a transportable liquid hydrocarbon product, comprising means for applying ultraviolet light to the natural gas to photodissociate methane therein and polymerize the same to C2+ hydrocarbons, and means for cooling the C2+ hydrocarbons to a sufficient extent whereby the transportable liquid hydrocarbon product is produced.

14. Apparatus as defined in claim 13, installed on an offshore oil and gas production platform.

15. Apparatus as defined in claim 13, installed on a floating production, storage and offloading vessel.
